(19) United States
(12) Patent Application Publication
(10) Pub. No.: US 2008/0214882 A1
(43) Pub. Date: Sep. 4, 2008

(54) ACIDIC MESOSTRUCTURED ALUMINOSILICATES ASSEMBLED FROM SURFACTANT-MEDIATED ZEOLITE HYDROLYSIS PRODUCTS

(75) Inventors: Thomas J. Pinnavaia, East Lansing, MI (US); Hui Wang, Des Plaines, IL (US); Yu Liu, Lake Jackson, TX (US)

Correspondence Address: HARNESS, DICKEY & PIERCE, P.L.C., P.O. BOX 828, BLOOMFIELD HILLS, MI, 48303, US

(73) Assignee: Board of Trustees of Michigan State University, East Lansing, MI (US)

(21) Appl. No.: 12/032,888

(22) Filed: Feb. 18, 2008

Related U.S. Application Data
(60) Provisional application No. 60/902,153, filed on Feb. 16, 2007.

(51) Int. Cl. C01B 39/02; C07C 4/06

(52) U.S. Cl. 585/653; 423/716

(57) ABSTRACT

The surfactant-mediated hydrolysis of ZSM-5 zeolite affords five-membered ring subunits that can be readily incorporated into the framework walls of a hexagonal mesostructured aluminosilicate, denoted MSU-Z. The five-membered ring subunits, which are identifiable by infrared spectroscopy, impart unprecedented acidity to the mesostructure, as judged by cumene cracking activity at 300° C. Most notably, MSU-Z aluminosilicate made through the base hydrolysis of ZSM-5 in the presence of cetyl trimethyl ammonium ions exhibits a cumene conversion of 73%, which is 6.7-fold higher than the conversion provided by a conventional MCM-41. This approach to stabilizing zeolitic subunits through surfactant-mediated hydrolysis of zeolites appears to be general. The hydrolysis of USY zeolite under analogous hydrolytic conditions also affords zeolitic fragments that boost the acidity of the mesostructure in comparison to equivalent compositions prepared from conventional aluminosilicate precursors.

### CROSS-REFERENCE TO RELATED APPLICATIONS

This application claims the benefit of U.S. Provisional Application No. 60/902,153 filed on Feb. 16, 2007. The disclosure of the above application is incorporated herein by reference.

### GOVERNMENT SUPPORT

The support of this research through NSF grant CHE-0211029 is gratefully acknowledged. The United States Government may have rights in this invention.

### BACKGROUND

The present teachings relate to acidic mesostructured aluminosilicate and methods for their synthesis from zeolites by surfactant mediated hydrolysis.

The high surface areas and large pore sizes of mesostructured aluminosilicates have been recognized as desirable properties for the catalytic processing of high molecular weight petroleum fractions. However, due to the absence of atomic order in the mesostructured framework, the compositions lack the desired hydrothermal stability and acidity. Zeolites generally have good acidity, but have less surface area and smaller pore sizes than mesoporous aluminosilicates.

Attempts to crystallize the amorphous walls of mesostructured aluminosilicates into zeolite have resulted in collapse of the mesostructure. Using two templates for the crystallization of both a microporous zeolite framework and a mesoporous structure ended up providing a mixture of separate phase. Several papers relate to the use of hydrolyzed zeolite products as precursors for mesostructure synthesis. However, when hydrolysis is conducted in absence of surfactant, little or no improvement in the acidity of the final mesostructure is achieved. Recently, Ying et al. US 2005/0239634 have reported a crystalline zeolite material organized in a mesostructure, which is prepared by adding a zeolite to an acid or base medium, adding a surfactant to the medium, optionally hydrothermally treating the medium, and washing and drying the resulting material. Surfactant can be removed by calcining. The mesostructure material produces high angle Bragg reflections indicating the presence of a crystalline zeolite phase.

Mesostructured derivatives with improved hydrothermal stability and acidity have been reported based on the incorporation of protozeolitic nanoclusters or “zeolite seeds” in the framework. This was accomplished by quenching a zeolite synthesis gel prior to the onset of zeolite crystal formation and then transforming the quenched gel into a mesostructure. The generality of this approach and the spectroscopic evidence supporting the presence of zeolitic subunits in the mesostructured framework has been substantiated by several subsequent studies.

Several United States patents describe a “bottom up” protozeolitic approach and include U.S. Pat. Nos. 6,585,952; 6,702,993; 6,706,169; 6,746,659; 6,770,258; 6,843,977; 6,869,906; and 7,128,892; the disclosures of which are incorporated by reference. In these patents, the pore walls of the mesostructure are amorphous but contain some zeolitic subunits.

Further efforts to produce mesostructured aluminosilicates having a combination of desirable properties, such as increased acidity for catalytic activity, are ongoing.

### SUMMARY

Aluminosilicates having a suitable level of acidity for use as hydrocarbon cracking catalyst are produced by surfactant-mediated transformation of zeolite into an aluminosilicate mesostructure with retention of zeolite structure and the associated acidity in the walls of the mesostructure. The product aluminosilicate is mesoporous, having pores of 2 nm to 50 nm in diameter; is mesostructured as determined by the existence of Bragg peaks in the X-ray pattern corresponding to d spacings of about 2 nm to 50 nm; possesses zeolite structure but does not exhibit high angle scattering from a crystalline zeolite phase; and is highly acidic, as indicated for example by an improved conversion of cumene compared to conventional mesoporous aluminosilicates. The Si/Al ratios for the mesostructured products are equivalent to those of the initial zeolite precursors. Also, after ammonium ion exchange, only a trace amount of Na+ is present in the mesostructure.

In one embodiment, the surfactant-mediated hydrolysis of ZSM-5 zeolite affords five-membered ring subunits that can be readily incorporated into the framework walls of a hexagonal mesostructured aluminosilicate, denoted MSU-Z. The five-membered ring subunits, which are identifiable by infrared spectroscopy, impart acidity to the mesostructure, as judged by cumene cracking activity at 300° C. MSU-Z aluminosilicate made through the base hydrolysis of ZSM-5 in the presence of cetyl trimethyl ammonium ions exhibits a cumene conversion of 73%, which is 6.7-fold higher than the conversion provided by a conventional MCM-41. This approach to stabilizing zeolitic subunits through surfactant-mediated hydrolysis of zeolites appears to be general. The hydrolysis of USY zeolite under analogous hydrolytic conditions also affords zeolitic fragments that boost the acidity of the mesostructure in comparison to equivalent compositions prepared from conventional aluminosilicate precursors.

The materials are made using a “top-down” approach to improving the incorporation of zeolitic subunits into the framework walls of a mesostructure based on the hydrolysis of a pre-formed zeolite in the presence of a surfactant. The hydrolysis is carried out at elevated pH. After hydrolysis, the pH is adjusted to a lower value suitable for formation of an aluminosilicate framework and the material is heated to form the mesostructured product. It is believed the surfactant stabilizes the zeolitic subunits during hydrolysis, and also serves as a structure-directing porogen for the assembly of the mesostructure.

This “top-down” approach to incorporating zeolitic subunits into the framework walls of a mesostructure substantially boosts the acidity of the mesostructure beyond the levels achievable through the “bottom-up” methodology based on the formation of protozeolitic seeds. Evidence for the effectiveness of this new approach to incorporating zeolitic subunits into a mesostructure framework is provided in some aspects by FTIR spectroscopy and by the activity of the mesostructures as cumene cracking catalysts.

The surfactant-mediated hydrolysis of ZSM-5 described in the present work is more effective than direct zeolite hydrolysis for the preparation of mesostructures with high levels of five ring subunits and high acid cracking activity. The improved cumene cracking activity observed for MSU-Z made by surfactant-mediated hydrolysis of USY zeolite also suggests that the “top-down” approach to the incorporation of zeolitic subunits into the framework walls of a mesostructure is general and most likely applicable to other zeolite precursors.

### BRIEF DESCRIPTION OF THE DRAWINGS

FIG. 1 shows N₂ adsorption/desorption isotherms for (A) conventional MCM-41 aluminosilicate, (B) MSU-Z prepared from hydrolyzed USY as a precursor, and (C) MSU-Z prepared from hydrolyzed ZSM-5 as a precursor. Curve B and C are offset by 200 and 400 for clarity.

FIG. 2 shows BJH pore size distributions obtained from adsorption isotherms for (A) conventional MCM-41 aluminosilicate, (B) MSU-Z prepared from hydrolyzed USY as precursor, and (C) MSU-Z prepared from hydrolyzed ZSM-5 as precursor.

FIG. 3 shows X-ray diffraction patterns for (A) conventional MCM-41 aluminosilicate, (B) MSU-Z prepared from hydrolyzed USY zeolite as a precursor, and (C) MSU-Z prepared from hydrolyzed ZSM-5 as a precursor.

FIG. 4 shows an X-ray diffraction pattern of MSU-Z prepared from an incompletely hydrolyzed ZSM-5 zeolite precursor. The expanded diffraction peaks are assigned to the un-hydrolyzed zeolite phase.

FIG. 5 shows FT-IR spectra of calcined mesostructured aluminosilicates (2 mg per 400 mg KBr) for (A) MCM-41 assembled from conventional precursors; (B) MSU-S assembled from protozeolitic ZSM-5 seeds; (C) MSU-Z assembled from zeolite fragments generated through the surfactant mediated hydrolysis of ZSM-5 zeolite in 0.45 M NaOH (OH−/T=0.75); and (D) pristine ZSM-5, respectively.

### DESCRIPTION

ATOMICALLY ORDERED or CRYSTALLINE: refers to a solid in which atoms are arranged on lattice points over a length scale effective in producing Bragg reflections in the wide angle region of the X-ray powder diffraction pattern of the solid.

WIDE ANGLE DIFFRACTION: refers to the Bragg diffraction features in the two theta region of an X-ray powder diffraction pattern corresponding to one or more basal spacings less than one nanometer in magnitude.

POROUS: refers to a solid containing open pores or space that can be accessed and occupied through sorptive forces by one or more guest species of molecular dimensions, the said space may be contained within a single particle of the solid or between aggregates of particles.

MESOPOROUS: refers to a porous solid in which the average pore size diameter is in the meso length scale range 2-50 nm.

MESOSTRUCTURED: refers to a structured form of a solid wherein the element of structure repeats on a length scale between 2-50 nm, resulting in the presence of at least one Bragg reflection in the small angle X-ray powder diffraction pattern of the solid. The repeating element of structure may be atomically ordered (crystalline) or disordered (amorphous).

SMALL ANGLE DIFFRACTION: refers to the Bragg diffraction features in the two theta region of an X-ray powder diffraction pattern corresponding to one or more basal spacings greater than one nanometer in magnitude.

In one embodiment, the teachings provide a mesostructured aluminosilicate material having a unique combination of structural parameters and catalytic activity. The products are mesoporous, exhibiting pores having a diameter from about 2 nanometers to about 50 nanometers. The products are also mesostructured, exhibiting Bragg reflections in the low angle portion of the x-ray diffraction spectrum corresponding to d spacings of 2-50 nm. Recognizable zeolitic subunits are incorporated into the walls of the mesoporous products, although a crystalline zeolite phase is not present. Accordingly, the X-ray diffraction pattern of the products shows no high angle Bragg reflections that would correspond to the crystalline zeolite phase.

In another embodiment, there are provided methods for transforming zeolites into acidic mesostructured materials. The methods comprise hydrolyzing the zeolite in the presence of a surfactant by heating an aqueous suspension of the zeolite at a pH of 11 or greater to make a hydrolysis product. Then the pH of the hydrolysis product is reduced to a lower pH below 11 and the acidic mesostructured material is assembled by heating at the lower pH. In various embodiments, the hydrolysis is mediated by heating in the presence of a cationic surfactant. As described further below, a preferable pH for the hydrolyzing step is 12 or greater, while for the assembling step is 10 or less. In various embodiments, the hydrolyzing and/or assembling step is carried out at a temperature of 80° C. or more, or around 100° C.

In another embodiment, the methods described herein transform crystalline zeolite to a mesostructured aluminosilicate having improved acidity, which improved acidity is believed to be due to the incorporation of zeolitic subunits into the walls of the mesostructures. The method involves surfactant mediated hydrolysis at a first pH above 11, followed by adjusting the pH and assembling the mesostructured materials by continuing the hydrolysis at a second pH below 11. In the methods described herein, it is believed that the surfactant present in the first hydrolysis step acts as a porogen in the assembling step following the reduction in pH.

In another embodiment, the current teachings provide a mesoporous aluminosilicate having structure to make it useful for example as a cracking catalyst. The mesoporous aluminosilicates are characterized by pores of 2 nm to 50 nm in diameter. They are further characterized by the presence in an x-ray diffraction pattern of Bragg peaks corresponding to d spacings of 2 nm to 50 nm. The x-ray diffraction pattern is further characterized by an absence of high angle scattering from a crystalline zeolite phase. Finally, the mesoporous aluminosilicates describes herein have a high acidity. In one aspect, the high acidity is evidenced by a conversion of 20% or greater in a cumene cracking reaction, when the reaction is carried out at 300° C. for 3 hours in a 6 mm id fixed quartz bed reactor using 200 mg of the mesoporous aluminosilicate as catalyst and at a cumene flow rate of4.1 μmol per minute with a nitrogen carrier gas of 20 cm³ per minute. In various embodiments, the acidity of the mesoporous aluminosilicates is marked by the achievement of conversion of 30% or greater in the cumene cracking reaction. In other embodiments, the conversion achieved matches or exceeds 50%, and in a preferred embodiment is about 70%-99%.

Methods are also provided of cracking hydrocarbons using the mesoporous aluminosilicates described herein. Thus in one embodiment, a method of cracking hydrocarbons comprises contacting the hydrocarbon with a cracking catalyst to obtain a cracking conversion of at least 20%, wherein the cracking catalyst is any of the high acidity mesoporous aluminosilicates described herein, and/or made by the two step methods described herein.

In one aspect, the products of the current teaching are characterized by X-ray diffraction. As noted, the X-ray diffraction pattern of the products exhibits the low angle peaks characteristic of mesostructured products, but exhibits no high angle peaks typical of zeolite crystalline phases. As developed below, in various aspects, the presence of high angle peaks in the diffraction pattern of the products made herein is an indication that incomplete hydrolysis has been carried out. In various embodiments, the observation of such diffraction peaks is taken as experimental evidence that the concentration of base, the concentration of surfactant, or both should be optimized during the hydrolysis procedure to provide complete hydrolysis where that is desired.

The products are also characterized by incorporation of zeolitic subunits into the walls of the mesoporous structures. Evidence for such incorporation lies in the observation of increased acidity in the structures. In several aspects, it is also confirmed by infrared spectroscopic measurements. Thus, it is possible to observe the incorporation of pentasil zeolite fragments in the walls of the mesoporous structure, even though the zeolite phase is non-crystalline as shown by the absence of high angle Bragg peaks. It is noted that the spectroscopic evidence of incorporation of the zeolitic subunits is available only for those zeolitic subunits that provide unique infrared bands such as the pentasil. However, it is believed that, even in the absence of the possibility of observing such an IR band in the products, the transformed zeolite products of the current teachings include such structures in their walls by analogy and by observation of an increased acidity.

In various embodiments, increased acidity in the products of the current teachings is inferred from the observation of improved catalytic cracking, as for example demonstrated by the cumene conversion experiments described herein. Thus, where improved catalytic activity is observed, it can be inferred that zeolitic subunits have been incorporated into the walls of the mesostructured products.

Experimentally, where the pentasil structure of the zeolite is present, the presence of the feature in the mesostructured products is determined by measuring the IR absorption intensity in the area around 550 cm⁻¹ and comparing to the intensity of the same peak in the starting zeolite material. The observed IR intensity in the 550 cm⁻¹ band is normalized by conventional means such as by comparing the intensity to the intensity of an IR absorbance band at 450 cm⁻¹, which is attributable to the Si-O-Si linkage present in all silica materials. In various embodiments, the normalized IR intensity of the band in the mesostructured product is at least 10% that of the band in the starting material. In preferred embodiments, the intensity is at least 20% and preferably at least 50% of the intensity of the band in the starting material, where intensity is measured as peak height. Such spectroscopic observations provide evidence that the zeolitic subunits of the starting zeolite are incorporated into the walls of the mesoporous products, even though a crystalline zeolite phase is absent, as determined by the absence of high angle Bragg peaks.

In various embodiments, methods are provided for transforming crystalline zeolite to mesostructured aluminosilicates having improved acidity by virtue of zeolitic subunits incorporated into the walls of the mesostructures. In one aspect, the method is carried out in two steps. The first is a surfactant-mediated hydrolysis of a crystalline zeolite product, and the second is an assembly of the zeolite hydrolysis products into an acidic mesostructure. For example in the first step, the zeolite crystal structure is broken down into zeolitic nanoclusters or zeolite fragments through basic hydrolysis in the presence of a surfactant. The hydrolysis is carried out at a suitable pH, but is normally at a pH value higher than the pH value needed to form an aluminosilicate mesostructure through a molecular assembly process. Normally the hydrolysis in the presence of the surfactant is carried out at a high pH, which is typically above pH 11 and more typically above pH 12. The second step, the assembly of zeolitic nanoclusters or fragments, is carried out at a lower pH value, typically below a pH value of 11 or 12, and more preferably below a pH of 10, while still maintaining a basic pH. Thus, in various embodiments, after the hydrolysis the pH of the medium is adjusted downward to, say, a pH of about 9 with an acid such as sulfuric acid.

During the hydrolysis step, the surfactant serves to stabilize the zeolite fragments and prevent them from being completely hydrolyzed. In this way, it is believed that more zeolitic subunits are preserved in the hydrolysis solution; which are subsequently available for conversion to the acidic mesostructure in the second step. Then, during assembly, at a lower pH, zeolitic crosslinking is incorporated into the framework walls of the mesostructure, leading to observance of high acidity in the mesostructure comparable to that of the parent zeolite.

Hydrolysis and assembly are carried out at elevated temperatures as desired to achieve suitable reaction completion. Preferably, the steps are carried out at elevated temperatures such as 80° C. and above. By use of an autoclave, temperatures above 100° C. are available to use with the aqueous systems. It has been found that 100° C. is a suitable temperature for the two processes.

Before hydrolysis, it is preferred to combine the surfactant and zeolite in an aqueous suspension and to mix the components thoroughly. Typically, such mixing is carried out at room temperature (or at a higher temperature, but one that avoids significant hydrolysis) with vigorous stirring. A stirring time of 60 minutes has been found to be acceptable. Following the mixing of a surfactant and the zeolite (the suspension normally also contains the base), the basic solution/suspension of surfactant, base, and zeolite is heated to a temperature such as 100° C. for a suitable time. A time of 24 hours at 100° C. has been found suitable to degrade the zeolite.

Following hydrolysis, the mixture is cooled to room temperature and the pH is adjusted through the addition of dilute acid such as sulfuric acid. As noted, the pH is adjusted preferably to below 11, preferably to 10 or below. It has been found suitable to adjust the pH to 9. The mixture is then exposed to an elevated temperature such as 100° C. for another period such as 24 hours to form the mesostructure. The product is then washed, for example with distilled water, it is air dried, and then calcined at an elevated temperature (for example about 600° C.) for a suitable time (for example for about 4 hours) to remove the surfactant. A protonated form of the resulting mesostructure can be obtained by ion exchange of the sodium with, for example, 0.5 molar ammonium nitrate solution (for example at 60° C. for 2 hours) prior to calcining.

The hydrolysis is carried out in the presence of base. The concentration of base is selected so as to achieve acceptable results of hydrolysis. The use of too low a base concentration can result in incomplete hydrolysis so that the final mesostructured form after the second step will be observed to contain a mixture of the mesostructured product of the current teachings as well as unreacted zeolite phase. For example, such incomplete hydrolysis can be detected so that zeolite peaks are observed in the high angle region of the X-ray diffraction pattern of the mesoporous product. On the other hand, too high a base concentration leads to over hydrolysis of the zeolite and diminished incorporation of zeolitic subunits into the walls of the mesoporous structure with concomitant loss of observed acidity as for example demonstrated by measurements of cumene conversion.

A suitable base concentration can be different depending on the nature of the starting zeolite. Examples of suitable levels for base hydrolysis of ZSM-5 zeolite and USY zeolite are provided in the current description. With other zeolites, such as without limitation zeolite Y and zeolite X, it may be necessary to carry out routine experimentation to determine the suitable range of base concentration for the hydrolysis.

Along these lines, it is noted that the total amount of base in a hydrolysis solution can be expressed relative to the amount of zeolite in the well known OH−/T ratio where T represents total Si plus Al. Thus, for ZSM-5 as a precursor, hydrolysis with an OH⁻/T ratio of 0.75 provides suitable hydrolysis (see Example 1) while hydrolysis at an OH⁻/T ratio of 0.58 showed evidence of incomplete hydrolysis (see Example 3). In addition, when hydrolysis of the ZSM-5 is carried out at an OH⁻/T ratio of about 1.5, there is evidence of over-hydrolysis with little incorporation of acidity into the mesostructure (see Example 4). Accordingly, hydrolysis of ZSM-5 zeolite is carried out above an OH⁻/T ratio of 0.58 and below an OH⁻/T ratio of 1.5, below an OH⁻/T ratio of 1.2, or below an OH⁻/T ratio of 1.0.

With another zeolite, suitable hydrolysis is observed at 0.1 M base and an OH⁻/T ratio of 0.17, well below the OH⁻/T of 0.56 observed to lead to partial hydrolysis with ZSM-5 (see Example 2).

In light of the above, the current teachings include carrying out hydrolysis at elevated pH in the presence of surfactant in a series of base concentrations and selecting a base concentration for hydrolysis to form an acidic mesostructure based on observation of one or more of acidity, IR spectrum, or X-ray diffraction pattern in the resulting mesostructure. A suitable base concentration is one that will result in improved acidity of the mesoporous structure, preferably along with incorporation of zeolitic subunits into the walls of the mesostructure and the absence of a separate zeolite crystalline phase in the mesoporous product.

In various embodiments, the concentration of surfactant affects the progress of the hydrolysis reaction. Accordingly, the surfactant concentration should be chosen in a range to give acceptable results. However, it is noted that the surfactant concentration has less effect than the base concentration discussed above. For this reason, it is believed that a wide range of surfactant concentrations are suitable. Nevertheless, it is preferred in various embodiments to determine a suitable level of surfactant, for example by carrying out hydrolysis at various concentrations of surfactant and choosing that surfactant concentration or range of concentrations that leads to the desired end product. For example, the end product resulting from hydrolysis in the presence of surfactant should exhibit increased acidity while spectroscopically there should be X-ray peaks observed in the low angle region and no X-ray peaks observed in the high angle region.

In various embodiments, combinations of mesostructured products as described herein and unreacted zeolite phase, resulting for example from incomplete hydrolysis as discussed above, are provided that, despite the presence of zeolite phase, still exhibit increased acidity when compared to mesoporous structures formed by other means. Thus, even though it is preferred in various embodiments to provide mesostructures having no independent zeolite phase, it is understood that mixtures of such mesostructured products with unreacted zeolite starting material can also provide improved catalysis based on the overall improved acidity and the presence of the mesopores which leads to high surface area.

Suitable surfactants include cationic, nonionic, and anionic surfactants. In various embodiments, cationic surfactants or nonionic surfactants are used. Non-limiting examples of cationic surfactants include cetyl trimethyl ammonium bromide, while a non-limiting example of a nonionic surfactant is an alkyl amine. Surfactants are used during the hydrolysis in suitable concentrations to form micelles effective at stabilizing zeolitic nanoclusters or zeolite fragments during the hydrolysis. After hydrolysis, and in the second step of framework assembly, the surfactant molecules serve a role similar to those of porogens in synthesis of conventional mesoporous structures by polymerization of gel starting materials.

The X-ray diffraction patterns of mesostructured materials assembled from ZSM-5 and USY zeolite fragments by the methods described herein both show hexagonal symmetry characteristic of mesoporous structure MCM-41, but have no Bragg peaks indicative of a zeolite phase. The absence of a zeolite phase is further shown by the inability to observe lattice fringes by transmission electron microscopy that would be indicative of atomic order. On that basis, it is believed that an authentic zeolite phase is absent from the structures. Further, the ²⁷AI MAS MNR chemical shifts of the as made mesostructured materials described herein (i.e., before calcination) indicate that all of the aluminum in the mesostructures is in tetrahedral coordination Upon calcination, some octahedral aluminum is observed at about 3 ppm, but the majority centers remain in tetrahedral coordination.

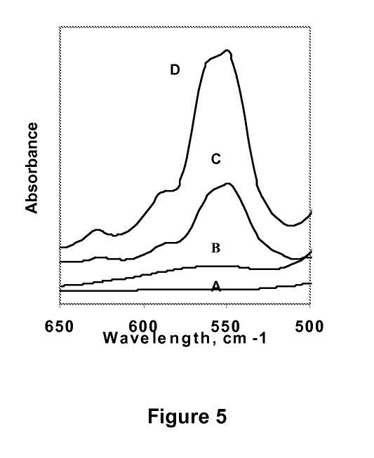

As noted, the presence of zeolitic structure in the mesostructured materials described herein can be observed in the infrared spectrum. As discussed further in the Examples below, FIG. 5 shows the intense absorbance of starting zeolite (curve D) compared to the relatively intense band C from the mesostructured material made by the process described herein. It is seen that the IR band of B at 550 cm⁻¹ is on the order of about 50% as intense as that of the starting zeolite. The figure shows that the IR absorbance of the starting zeolite and the mesostructured material made from it are much more intense than mesoporous materials made in conventional fashion or in bottom-up assembly from protozeolitic seeds.

In various aspects, the mesoporous aluminosilicates prepared by the methods described here in exhibit an advantageous high acidity, which makes them useful catalysts for hydrocarbon cracking. As further discussed herein, the cracking is believed to be due to the presence of high acidity in the catalyst materials, and indeed the high acidity properties of the materials can be inferred from their enhanced catalyst cracking reactivity. In general, advantageous high acidity in the materials is evidenced by the materials exhibiting high cumene conversions compared with conventional aluminosilicates and zeolites that do not have the novel combination of structural features of the materials prepared here. In various embodiments, the mesoporous aluminosilicates of the invention are characterized by cumene conversions that are greater than 11%, and that approach conversions of 90-95% or higher. Thus in various embodiments, the cumene conversion achieved by use of the mesoporous aluminosilicates of the inventions as catalysts is 20% or greater, 30% or greater, 50% or greater, or about 70%-99%. As shown for example in Table 1 below, cumene conversion is calculated from results of a cumene cracking reaction carried out at 300° C. in a 6 mm id fixed bed quartz reactor using 200 mg of the mesoporous aluminosilicate as catalyst. For purposes of comparison, the cumene conversion is measured at a cumene flow rate of 4.1 μmol per minute using nitrogen carrier gas at 20 cm³/minute. The reaction is carried out for 3 hours and the percent conversion of cumene is calculated.

The invention has been described above with respect to various exemplary and/or preferred embodiments. Further non-limiting description is found in the examples that follow.

### EXAMPLES

### Example 1

This example illustrates the hydrolysis of ZSM-5 zeolite in ammonium ion exchanged form and the use of the hydrolyzed product as a precursor for the assembly of a mesostructured MSU-Z aluminosilicate. Ammonium exchanged ZSM-5 was obtained from Zeolyst (CBV 8040) with a Si/Al ratio of 40.

ZSM-5 zeolite (2.00 g, 33.3 mmol) was dispersed in a solution containing 55 mL of 0.45 M NaOH solution (OH⁻/T=0.75, where T=Si +Al), to which 2.45 g (6.7 mmol) cetyl trimethyl ammonium bromide (CTAB) was added as porogen. The mixture was stirred at room temperature for 1 hour, and then transferred into a Teflon-lined autoclave, heated at 100° C. for 24 hours, and then cooled to room temperature. The pH of the solution was adjusted to 9.0 through the addition of sulfuric acid and the mixture was heated again at 100° C. for another 24 hours. The product was filtered, washed, and calcined at 600° C. for 4 hours. The final product was denoted MSU-Z. The protonated form of MSU-Z was obtained by duplicate ion-exchange reaction with 0.5 M NH₄NO₃ solution at 60° C. for 2 hours and then calcining the product at 550° C.

### Example 2

This example illustrates the hydrolysis of USY zeolite and the use of the hydrolyzed product as a precursor for the assembly of a mesostructured MSU-Z aluminosilicate. USY zeolite is a steamed version of zeolite Y. The steam treatment leaches aluminum from the framework and improves acid strength and hydrothermal stability. Zeolite Y is a high silica analog of zeolite X. All three zeolite forms possess a faujasite framework structure. Thus, any one of the zeolites is a suitable precursor for the synthesis of MSU-Z mesostructures. USY zeolite was obtained from Zeolyst (CBV 720) with a Si/Al ratio of 40. The USY zeolite (2.00 g, 33.3 mmol) was dispersed in 55 mL of 0.10 M NaOH solution (OH⁻/T=0.17), to which cetyl trimethyl ammonium bromide (CTAB, 2.45 g, 6.7 mmol) was added as a porogen. The mixture was stirred at room temperature for 1hour, transferred into a Teflon-lined autoclave, heated at 100° C. for 24 hours and then cooled to room temperature. The pH of the solution was adjusted to 9.0 through the addition of sulfuric acid. The mixture was again heated at 100° C. for another 24 hours to obtain the mesostructured product. The product was converted to the proton exchange form by exchange reaction with ammonium nitrate and calcination, as described in Example 1.

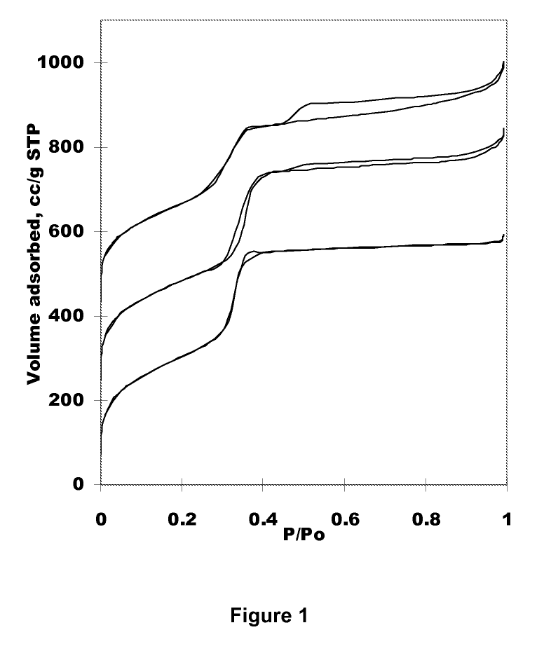

FIG. 1 provides the N₂ adsorption/desorption isotherm of MSU-Z compositions in comparison to a conventional MCM-41 aluminosilicate mesostructure (Si/Al=40), which was prepared from sodium aluminate and sodium silicate solution as alumina and silica sources.

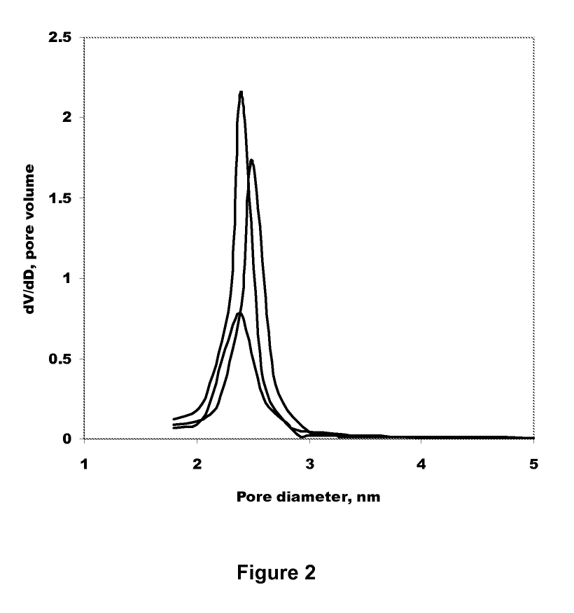

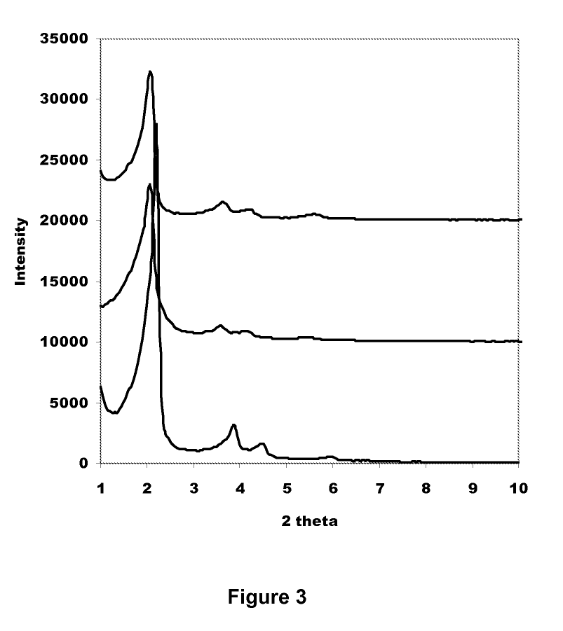

FIG. 2 provides the pore size distribution of MSU-Z samples. FIG. 3 provides the X-ray powder diffraction patterns. These results show that MSU-Z materials are high quality mesostructures.

### Example 3

This example illustrates the possibility of synthesizing a composite of mesostructured and zeolite phases when insufficient NaOH was used as a zeolite hydrolysis reagent.

ZSM-5 (2.00 g, 33.3 mmol) was dispersed in 55 mL of a solution containing 0.75 g NaOH (OH⁻/T=0.58) and 2.45 g cetyl trimethyl ammonium bromide (CTAB). The mixture was stirred in room temperature for 1hour, transferred to a Teflon-lined autoclave, heated at 100° C. for 24 hours and then cooled to room temperature. The pH of the solution was adjusted to 9.0 through the addition of sulfuric acid. The mixture was again heated at 100° C. for another 24 hours. The product was converted to the proton exchange form by exchange reaction with ammonium nitrate and calcination, as described in Example 1.

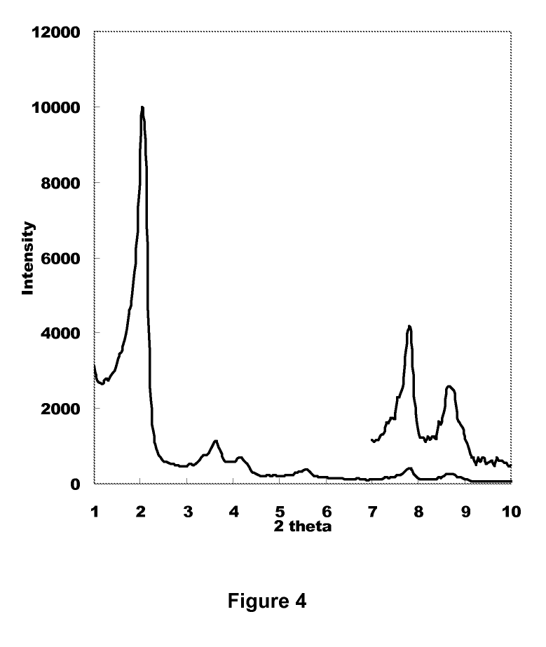

The X-ray diffraction pattern of the product provided in FIG. 4 exhibits the low angle reflections for a hexagonal mesostructure and the wide angle reflections indicative of the presence of the incompletely hydrolyzed zeolite phase.

It is intended that the foregoing description be only illustrative of the present invention and that the present invention be limited only by the hereinafter appended claims.

### Example 4

Even in the presence of the surfactant, however, it is possible to over-hydrolyze the zeolite fragments. For instance, increasing the NaOH concentration in Example 1 for ZSM-5 hydrolysis to 0.91 M (OH⁻/T ratio of about 1.5) affords a mesostructure with no 550 cm⁻¹ band and little improvement in cumene conversion compared to MCM-41 (cf. Table 1).

### Example 5

As shown in FIG. 5, the IR spectrum of MSU-Z formed from ZSM-5 fragments (curve C) exhibits an intense band near 550 cm⁻¹, indicative of five-membered ring subunits analogous to those subunits found for the pristine zeolite (curve D). This band is absent in the spectrum of MCM-41 prepared from conventional aluminosilicate precursors (curve A) and comparatively weak in the spectrum of MSU-S made from protozeolitic ZSM-5 seeds (curve B). As expected, this band is absent from the spectrum of MSU-Z made from hydrolyzed USY zeolite, as this zeolite does not contain five-ring subunits. On the basis of these FTIR results for MSU-Z made from hydrolyzed ZSM-5 fragments, it is clear that the “top-down” degradation of a zeolite in the presence of a fragment-stabilizing surfactant is superior to our earlier “bottom-up” approach to generating zeolite subunits from conventional aluminosilicate precursors.

### Example 6

To test the acidity of MSU-Z mesostructures (designated as MSU-Z) made from surfactant-mediated zeolite hydrolysis products; cumene cracking was carried out as a probe reaction. The results are included in Table 1. For comparison, cumene cracking also was carried out over ammonium exchanged conventional MCM-41, which showed a cumene conversion of 11%, consistent with previously reported results. The MSU-Z mesostructure prepared from USY fragments (Example 2) showed a substantial 3.4-fold increase in cumene conversion to in comparison to MCM-41. This high cumene activity value is similar to the activity observed for MSU-S aluminosilicates assembled from faujasite zeolite seeds. The 73% conversion found for MSU-Z made from the surfactant mediated hydrolysis of ZSM-5 (Example 1) is 6.7-fold higher than observed for a conventional MCM-41.

### COMPARATIVE EXAMPLES

Several composite compositions containing mixtures of zeolite and mesostructured aluminosilicate phases have been reported previously. These mixtures show wide angle Bragg X-ray reflections characteristic of the zeolite phase. Also, the zeolite component of these mixtures typically exhibits lattice fringes in the bright field TEM mode and dense nanocrystallite domains in dark field mode. None of these XRD or TEM features representative of a zeolite phase were observed for MSU-Z made from surfactant-mediated ZSM-5 fragments. Moreover, the absence of zeolitic microporosity was verified through t-plots of the nitrogen adsorption data, as expected for a framework that contains the secondary building units of a zeolite, but not zeolite cavities.

When the hydrolysis of ZSM-5 was carried out in the absence of surfactant and the pH adjusted to 9.0, a low surface area aluminosilicate gel was obtained which had no 550 cm⁻¹ IR band and virtually no activity for cumene cracking.

We note that MCM-41 aluminosilicates containing zeolitic fragments have been assembled from solutions formed through the partial hydrolysis of ZSM-5 in the absence of a surfactant. This approach not only makes incomplete use of the zeolite but also affords a mesostructure with a Si/Al ratio that is 30-fold larger than the initial zeolite. Moreover, the incorporation of five-membered ring subunits into the framework walls of the mesostructure, as well as the cracking activity of the mesostructure, is no better than what can be achieved through the direct assembly of aluminosilicate mesostructures from protozeolite seeds formed in the presence of a zeolite template.

TABLE 1

| | | Si/Al | NaOH | | | | | |
|---|---|---|---|---|---|---|---|---|
| | Si/Al Zeolite | mesostructured | concentrated^{a} | unit cell | BJH pore | BET surface | pore vol. | cumene |
| mesostructure | precursor | Product | (mol/L) | size((nm) | size (nm) | area (m²/g) | (cm³/g) | conv.^{b} (%) |
| MCM-41 | | 50 | | 4.64 | 2.4 | 1135 | 0.87 | 11 |
| (conventional) | | | | | | | | |
| MSU-Z from | 40 | 42 | 0.45 | 4.98 | 2.4 | 1160 | 0.86 | 74 |
| ZSM-5 | | | | | | | | |
| MSU-Z from USY | 40 | 43 | 0.10 | 4.98 | 2.5 | 1051 | 0.86 | 37 |
| Mesostructure | 40 | | 0.91 | 4.75 | 2.2 | 1114 | 0.73 | 17 |
| formed from | | | | | | | | |
| over-hydrolyzed | | | | | | | | |
| ZSM-5 | | | | | | | | |

^{a}Concentration of base used to hydrolyze the zeolite precursor.
^{b}Cumene cracking reaction was carried out at 300° C. in a 6 mm i.d. fixed bed quartz reactor; 200 mg catalyst; cumene flow rate, 4.1 μmol/min; N₂ carrier gas, 20 cm³/min; time-on-stream 3 h

The cumene cracking activity of MSU-Z made from surfactant-mediated ZSM-5 fragments approaches the 90-95% conversions observed for the pristine ZSM-5 at the same Si/Al ratio. This high acidity cannot be attributed to the presence of residual ZSM-5 crystals in the mesostructured product, because the presence of such crystals is precluded by the absence of wide angle Bragg peaks in the XRD powder pattern and the lack of TEM lattice fringes.

What is claimed is:

1. A method of transforming a zeolite starting material into an acidic mesostructured material, the method comprising
hydrolyzing the zeolite in the presence of a surfactant by heating an aqueous suspension of the zeolite in the surfactant at a pH above 11 to make a hydrolysis product;
adjusting the hydrolysis product to a lower pH below 11; and
assembling the acidic mesostructured material from the hydrolysis product by heating for a further time at the lower pH.

2. A method according to claim 1, wherein the surfactant is a cationic surfactant.

3. A method according to claim 1, comprising hydrolyzing the zeolite at a pH of 12 or greater.

4. A method according to claim 1, comprising assembling the mesostructured material at a basic pH of 10 or less.

5. A method according to claim 1, wherein the zeolite is ZSM-5 zeolite and the OH⁻/T ratio in the hydrolyzing step is about 0.75.

6. A method according to claim 1, wherein the zeolite is ZSM-5 zeolite and the OH⁻/T ratio in the hydrolyzing step is greater than 0.58 and less than 1.5.

7. A method according to claim 1, comprising hydrolyzing at a temperature of 80° C. or higher and assembling the mesostructured material by heating at a temperature of 80° C. or higher.

8. A method according to claim 1, wherein the hydrolyzing and assembling steps are carried out at a temperature of about 100° C.

9. A method according to claim 1, comprising hydrolyzing at a pH of 12 or higher at a temperature above 80° C., adjusting the pH to 10 or lower, and assembling at a temperature above 80° C.

10. A method for transforming crystalline zeolite to mesostructured aluminosilicates having improved acidity by virtue of zeolitic subunits incorporated into the walls of the mesostructures, the method comprising
surfactant mediated hydrolyzing the crystalline zeolite at a first pH of 11 or greater, followed by
adjusting the pH to a second pH below 11, and
assembling the mesostructured material by heating for a further time at a the second pH.

11. A method according to claim 10, wherein the first pH is 12 or greater and the second pH is 10 or less.

12. A method according to claim 10, wherein hydrolyzing comprises heating an aqueous suspension of the zeolite in the presence of the surfactant at a temperature of at least 80° C.

13. A method according to claim 12, wherein the temperature is about 100° C.

14. A method according to claim 12, wherein the zeolite is ZSM-5 zeolite and the OH⁻/T ratio in the hydrolyzing step is greater than 0.58 and less than 1.5.

15. A method according to claim 14, wherein the OH⁻/T ratio is about 0.75.

16. A method according to claim 10, wherein the surfactant is a cationic surfactant.

17. A method according to claim 10, wherein the surfactant is a nonionic surfactant.

18. A method according to claim 10, wherein the hydrolyzing and assembling steps are carried out at a temperature of about 100° C.

19. A method according to claim 10, wherein the zeolite is selected from the group consisting of USY zeolite, zeolite Y and zeolite X.

20. A mesoporous aluminosilicate, characterized by;
pores of 2 nm to 50 nm in diameter;
the presence, in an x-ray diffraction pattern taken of the mesoporous aluminosilicate, of Bragg peaks corresponding to d spacings of 2 to 50 nm;
an absence, in an x-ray diffraction pattern taken of the mesoporous aluminosilicate, of high angle scattering from a crystalline zeolite phase; and
a high acidity, wherein the high acidity is evidenced by a conversion of 20% or greater in a cumene cracking reaction carried out at 350° C. for 3 hours in a 6 mm id fixed bed quartz reactor with 200 mg of the mesoporous aluminosilicate at a cumene flow rate of 4.1 μmol per minute with nitrogen carrier gas at a flow rate of 20 cm³ per minute.

21. A mesoporous aluminosilicate according to claim 20, characterized by an acidity that results in a cumene conversion of 30% or greater.

22. A mesoporous aluminosilicate according to claim 20, characterized by an acidity that results in a cumene conversion of 50% or greater.

23. A mesoporous aluminosilicate according to claim 20 characterized by an acidity that results in a cumene conversion of 70%-99%.

24. A method of cracking hydrocarbons comprising contacting the hydrocarbon with a cracking catalyst to achieve a cracking conversion of at least 20%, wherein the catalyst is a mesoporous aluminosilicate, the mesoporous aluminosilicate characterized by:
pores of 2 nm to 50 nm in diameter;
the presence, in an x-ray diffraction pattern taken of the mesoporous aluminosilicate, of Bragg peaks corresponding to d spacings of 2 to 50 nm; and
the absence, in an x-ray diffraction pattern taken of the mesoporous aluminosilicate, of high angle scattering from a crystalline zeolite phase.

25. A method according to claim 24, wherein the method achieves a cracking conversion of at least 30%.

26. A method according to claim 24, wherein the method achieves a cracking conversion of at least 50%.

27. A method according to claim 24, wherein the method achieves a cracking conversion of 70%-99%.

28. A method according to claim 24, wherein the catalyst is made by a process comprising the steps of
hydrolyzing the zeolite in a surfactant by heating an aqueous suspension of the zeolite at a pH above 11 to make a hydrolysis product;
adjusting the hydrolysis product to a lower pH below 11; and
assembling the acidic mesostructured material from the hydrolysis product by heating for a further time at the lower pH.

29. A method according to claim 24, wherein the catalyst is made by a process comprising the steps of:
surfactant mediated hydrolyzing the crystalline zeolite at a first pH of 11 or greater followed by
adjusting the pH to a basic pH below 11 and assembling the mesostructured material by heating for a further time at the basic pH below 11.
